(19) United States
(12) Patent Application Publication
(10) Pub. No.: US 2024/0161909 A1
(43) Pub. Date: May 16, 2024

(54) ASSESSING MEDICAL PROCEDURES FOR COMPLETENESS BASED ON MACHINE LEARNING

(71) Applicant: International Business Machines Corporation, Armonk, NY (US)

(72) Inventors: Giovanni John Jacques Palma, Velizy Villacoublay (FR); Sun Young Park, San Diego, CA (US); Dustin Michael Sargent, San Diego, CA (US)

(21) Appl. No.: 17/984,566

(22) Filed: Nov. 10, 2022

(51) Int. Cl. G16H 40/20; G16H 50/20

(52) U.S. Cl. CPC G16H 40/20; G16H 50/20

(57) ABSTRACT

A present invention embodiment assesses medical procedures for completeness. Input data is received corresponding to a patient having a medical procedure. A plurality of predicted user actions to be performed during the medical procedure are determined. A plurality of actual user actions performed during the medical procedure are compared to the plurality of predicted user actions to identify one or more deviations in the medical procedure. A user is alerted to the one or more deviations.

### BACKGROUND

### 1. Technical Field

Present invention embodiments relate to machine learning, and more specifically, to assessing medical procedures for completeness based on machine learning.

### 2. Discussion of the Related Art

When a clinician assesses the condition of a patient, several steps are performed in order to assess all critical aspects of the patient's condition. In the field of radiology, for example, a radiologist may review several images of a patient, including views that may be specific to the radiologist. However, clinicians must frequently cope with interruptions during an assessment, such as other cases that require a higher priority, discussions with patients, and other external requests that can disrupt a clinician's workflow. These interruptions can have a negative impact on a clinician's reading and accordingly, a patient's outcome, as critical steps of an assessment can be missed.

### SUMMARY

According to one embodiment of the present invention, a system is provided for assessing medical procedures for completeness based on machine learning. Input data is received corresponding to a patient having a medical procedure. A plurality of predicted user actions to be performed during the medical procedure are determined. A plurality of actual user actions performed during the medical procedure are compared to the plurality of predicted user actions to identify one or more deviations in the medical procedure. A user is alerted to the one or more deviations. Embodiments of the present invention further include a method and computer program product for assessing medical procedures for completeness based on machine learning in substantially the same manner described above.

### BRIEF DESCRIPTION OF THE DRAWINGS

Generally, like reference numerals in the various FIGURES are utilized to designate like components.

FIG. 1 is a diagrammatic illustration of an example computing environment according to an embodiment of the present invention.

FIG. 2 is a block diagram of procedure assessment code according to an embodiment of the present invention.

FIG. 3 is a diagrammatic illustration of an environment for performing a medical procedure according to an embodiment of the present invention.

FIG. 4 is a flowchart of a method for assessing a medical procedure for completeness according to an embodiment of the present invention.

FIG. 5 is a flowchart of a method for training and applying a clustering model according to an embodiment of the present invention.

FIG. 6 is a flowchart of a method for training and applying a classification model according to an embodiment of the present invention.

### DETAILED DESCRIPTION

A present invention embodiment relates to assessing medical procedures for completeness based on machine learning. When a clinician performs a particular procedure, such as examining radiological images of a patient, the clinician performs a series of steps that may be necessary to fully assess the patient's condition. However, during the procedure itself, a busy clinician may be subjected to various distractions, such as answering messages, interacting with the patients themselves, switching to examine other patient records, and the like. Accordingly, present invention embodiments include techniques for determining the tasks that should be performed during a particular procedure, and monitoring the procedure itself to ensure that the clinician does not deviate from any necessary tasks. In some embodiments, a first computing model identifies tasks for a procedure, which can be universally required but also may be specific to a particular clinician, and a second computing model monitors the procedure to identify any potential deviations. The clinician can be alerted, either after the procedure or during the procedure (e.g., in real-time), so that any deviations can be addressed in order to minimize clinical error.

Accordingly, present invention embodiments improve the field of clinical medicine by providing the practical application of reducing errors in clinical procedures. Errors can be identified before a procedure is complete, thereby saving time and reducing costs to remedy any such errors, as the errors can instead be addressed while the procedure is still being conducted. Moreover, the computing model or models that identify the tasks that are involved in a procedure and/or that identify any deviations in a procedure can be updated based on user feedback (e.g., outcomes of patients, clinician feedback) to improve the models over time. Thus, present invention embodiments can reduce clinical errors in a manner that is itself improved over time, leading to better patient health care outcomes, reduced costs, more efficient and effective medical examinations, and the like.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

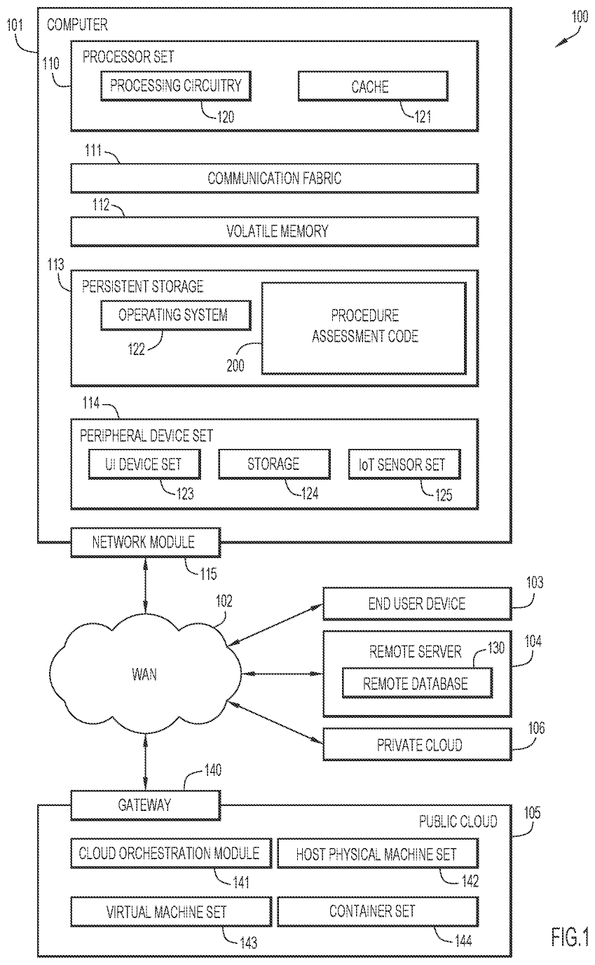

Referring to FIG. 1, computing environment 100 contains an example of an environment for the execution of at least some of the computer code involved in performing the inventive methods, such as procedure assessment code 200. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made though local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

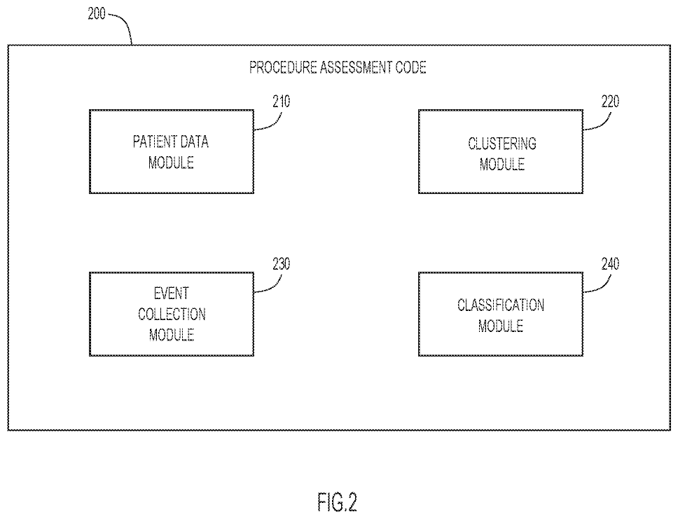

A block diagram of procedure assessment code 200 according to an embodiment of the present invention is illustrated in FIG. 2. Specifically, procedure assessment code 200 includes a patient data module 210, a clustering module 220, an event collection module 230, and a classification module 240.

Patient data module 210 obtains data relating to a patient and/or other data in order to support clinical tasks in accordance with present embodiments. In particular, patient data module 210 may obtain electronic health records of a patient who is the subject of a procedure. The patient data may include images of the patient's body, including images obtained via x-ray imaging, magnetic resonance imaging, ultrasound imaging, positron emission tomography, and the like. Patient data module 210 may pre-process the data by extracting a subset of data that is relevant for the procedure, such as International Classification of Diseases (ICD) codes in a patient's record, Digital Imaging and Communication in Medicine (DICOM) tags, and/or any other classifications or descriptions of a patient's medical condition(s). In some embodiments, patient data module 210 obtains other data entered by a health care provider, such as free-form text (e.g., clinical notes). Patient data module 210 may perform natural language processing tasks using a trained natural language processing model to extract topics from free-form text. Thus, patient conditions can be identified based on the presence of medical codes, descriptions by a health care provider, or a combination thereof.

Clustering module 220 processes data obtained by patient data module 210 in order to identify one or more user actions (e.g., actions performed by a clinician) that should be, or are likely to be, performed during the procedure. In various embodiments, clustering module 220 may employ a statistical model or machine learning model in order to identify mappings between patient data and user actions for a procedure. In some embodiments, a name of the procedure (e.g., “thoracic x-ray”) and/or an identity of the clinician (e.g., “Doctor Smith”, or any clinician that is a member of a particular practice group, etc.) may be provided to the model in order to identify the user actions for the procedure. Thus, clustering module 220 can identify user actions that are specific to a type of procedure, a disease or other medical condition of a patient, a particular practitioner, and/or any combinations thereof.

In some embodiments, clustering module 220 employs a statistical model, such as a probabilistic parametrization model; in other embodiments, clustering module 220 employs a machine learning model, such as a deep learning model. A deep learning model can be trained using examples of user actions that are performed for given examples of procedure types, patient data, and/or clinician identities. In some embodiments, the clustering algorithm employed by clustering module 220 extracts features from the medical images of the user and/or from DICOM tags in order to identify a set of user actions associated with the procedure. For example, given an input of thoracic x-rays, clustering module 220 may determine a series of user actions to be performed during the course of reviewing images (e.g., switching to different images, window width (WW) changes, window level (WL) changes, user dwell time on each view or image, coverage of computer tomography slices obtained, gimbal rotation settings, gimbal angle settings, magnification settings, contrast adjustment, measurements obtained, images or views of images saved for subsequent review, and the like). Clustering module 220 may employ a first machine learning model that is trained to identify features in medical images (e.g., organs, tissue, bones, particular views thereof, etc.) and a second machine learning model that is trained to map sets of identified features to sets of user actions. In some embodiments, a contrastive learning model is employed, which is trained using unlabeled examples of image data to learn how to identify features in input image data. Supervised or unsupervised training can be performed in one phase prior to applying the model, or can be performed continuously to improve the model over time.

In some embodiments, the model is updated based on user feedback that indicates whether the suggested user actions were correct or incorrect. Thus, a model of clustering module 220 can be continuously refined to strengthen associations between input and correct output and to weaken associations between input and incorrect output, thereby increasing the accuracy of the model over time.

Event collection module 230 monitors actual user actions performed by a clinician during a procedure (e.g., review of medical images, image-guided surgery, etc.) and collects data that classification module 240 can process to identify any deviations from the predicted user actions that are determined by clustering module 220. In some embodiments, event collection module 230 receives or obtains data from a computing device that is operated by a clinician during a procedure. Log data from a computing device operated by a clinician may store user actions performed by the clinician, such as zoom/pan operations, dwell time at certain views, and any other features associated with a procedure. Additionally or alternatively, one or more cameras may capture image data of a clinician performing a procedure on a patient, and an image processing model can be trained to identify user actions from images collected by the camera.

Classification module 240 compares predicted user actions for a procedure to actual user actions (e.g., collected by event collection module 230) in order to identify any deviations from the procedure. In some embodiments, classification module 240 may utilize a rules-based approach in which a deviation is identified based on predefined criteria, such as one or more predicted user actions not being performed by a user, or one or more predicted user actions not being performed within a threshold amount of time (e.g., for dwell time actions). In various embodiments, classification module 240 may utilize a probabilistic parametrization model or a deep learning model that is trained to identify any deviations in the procedure.

In an embodiment in which classification module 240 utilizes a deep learning model, the model may be trained using example sets of predicted user actions and actual user actions that are labeled to indicate whether the actual user actions represent a deviation or not. Thus, a conventional or other classifier can be trained and applied to process input comprising predicted user actions and actual user actions, and can indicate whether a deviation has occurred. The deep learning model of classification module 240 can be updated based on user feedback that indicates whether a particular classification was correct or incorrect, thereby enabling classification module 240 to update its deep learning model over time to improve the accuracy of classifications.

Classification module 240 may perform classifications in real-time during the course of a procedure to identify any deviations when the deviations occur (e.g., as opposed to after the conclusion of the procedure). Classification module 240 can alert a user (e.g., a clinician or other user) when a deviation occurs, so that the deviation can be remedied in an expedient manner.

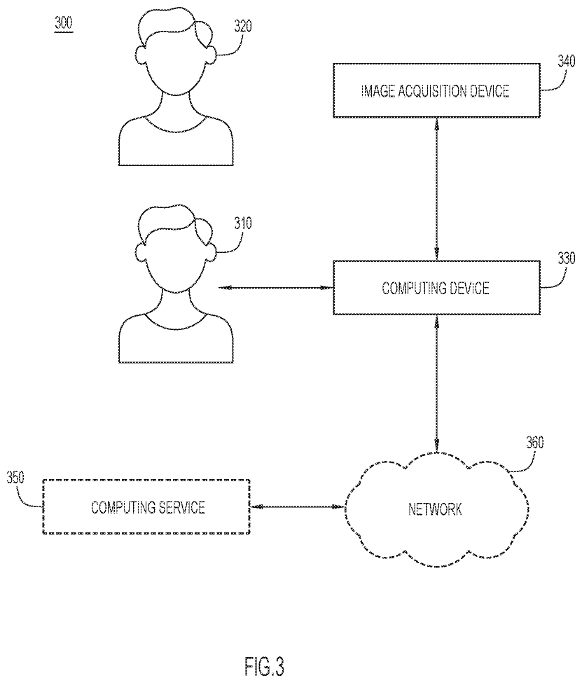

With reference now to FIG. 3, a diagrammatic illustration is shown of an environment 300 for performing a medical procedure according to an embodiment of the present invention.

As depicted, environment 300 includes a clinician 310, a patient 320, a computing device 330, an image acquisition device 340, and optionally, a computing service 350 and network 360. Computing device 330, image acquisition device 340, and/or computing service 350 may be implemented by any conventional or other computing device, such as computer 101, that is depicted and described in further detail with reference to FIG. 1. In some embodiments, computing device 330 and/or image acquisition device 340 may include, or be integrated into, a medical device, such as an x-ray imaging device, magnetic resonance imaging device, sonography device, and the like.

In various embodiments, clinician 310 may be performing any medical procedure with respect to patient 320. By way of example, a medical procedure can include, but is not limited to, an imaging procedure (e.g., x-ray imaging, magnetic resonance imaging, computerized tomography, positron emission tomography, sonography), image guided surgery (e.g., heart catheterization, neurosurgery, orthopedic surgery, urological surgery, an endoscopic procedure, etc.), or any other medical procedure (e.g., a dental procedure, an orthodontic procedure, etc.). The imaging may be performed prior to the surgery, during the procedure, or a combination thereof. Accordingly, in some embodiments, patient 320 may not be physically present when clinician 310 performs the procedure (e.g., in the case of post hoc review of images acquired at an earlier time).

During the procedure, clinician 310 may interact with computing device 330 to view and otherwise interact with images acquired of patient 320. For example, clinician 310 may perform window change operations (e.g., window width changes, window level changes, etc.), may scroll through different images, may pause or save live video to view or save still images, may scroll through two-dimensional slices of three-dimensional image data, may dwell on images for a certain amount of time, etc. In some embodiments, clinician 310 may use computing device 330 to control image acquisition device 340 (which can include any conventional or other medical imaging device) to cause image acquisition device to acquire images. For example, computing device 330 may send commands to image acquisition device 340 to control a gimbal rotation, position of robotic x-ray camera (e.g., pan, tilt, and/or zoom operations), or change any other parameter of image acquisition device 340 (e.g., a power setting, intensity setting, etc.).

In some embodiments, image acquisition device 340 includes one or more cameras that capture images of clinician 310 performing a procedure. These images can be provided to an image processing model that can extract user actions from the images. In particular, the images can be used to identify actual user actions, which can be compared to predicted user actions in accordance with present embodiments in order to determine whether there is a deviation in the procedure.

In some embodiments, computing device 330 includes components (e.g., patient data module 210, clustering module 220, event collection module 230, and/or classification module 240) to enable computing device 330 to independently assess a procedure for completeness. In other embodiments, computing service 350 includes some or all of the components (e.g., patient data module 210, clustering module 220, event collection module 230, and/or classification module 240) that are responsible for performing computing tasks to assess a procedure for completeness, and data is exchanged between computing device 330 and computing service 350 via network 360 in order to assess a procedure.

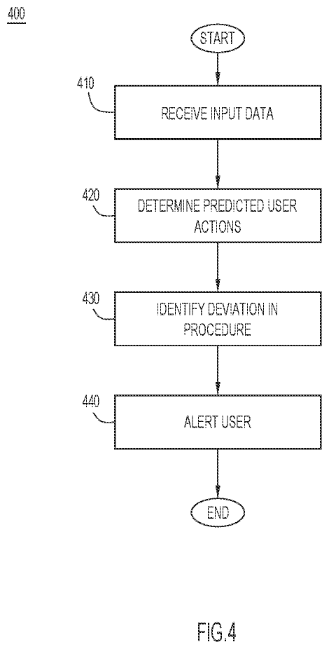

FIG. 4 is a flowchart of a method 400 for assessing a medical procedure for completeness according to an embodiment of the present invention.

Input data is received at operation 410. The input data can include any data describing a patient, such as electronic health records, medical imaging data of the patient, clinical notes regarding the patient, metadata, and/or any other data. The input data may be obtained from a database or other storage device, and can be obtained prior to a procedure or during a procedure. For example, the input data may include images that are acquired during a procedure.

Predicted user actions for a procedure are determined at operation 420. A computerized model may process the input data to identify one or more predicted user actions that a clinician may perform during the procedure. The model can include a statistical model or a machine learning model (e.g., a clustering model). The machine learning model may include any conventional or other machine learning models (e.g., mathematical/statistical, classifiers, feed-forward, recurrent, convolutional, deep learning, or other neural networks, etc.). The model identifies predicted user actions, which can include any input that a clinician provides to a computing device during the course of the procedure. In particular, the input can include any actions that the clinician performs while reviewing images of a patient, such as window changes, zooming, switching to other images, dwelling on a particular view for a threshold amount of time, and the like. In some embodiments, the predicted user actions are ordered in a particular order.

A deviation is identified in the procedure at operation 430. Actual user actions are captured during the procedure as the clinician interacts with a computing device to review medical images of the patient. The actual user actions may be obtained from a log on the computing device, and can be obtained during and/or after the procedure. In some embodiments, one or more cameras may capture image data of the clinician reviewing medical images, and one or more image processing models may analyze the captured image data to identify actions performed by the clinician.

The actual user actions may be compared to the predicted user actions in order to identify that a deviation has occurred in the procedure. A statistical model or deep learning model may be employed to identify deviations in the procedure by processing as input both the predicted user actions and the actual user actions. The machine learning or deep learning model may include any conventional or other machine learning models (e.g., mathematical/statistical, classifiers, feed-forward, recurrent, convolutional, deep learning, or other neural networks, etc.). In some embodiments, a deep learning model is trained to classify a procedure as having a deviation by determining when actual user actions deviate from predicted user actions by a machine-learned threshold amount. For example, if a clinician's actual dwell time is below a predicted dwell time for a particular image, a deviation may have occurred.

The user is alerted at operation 440. When a deviation is observed, the clinician may be alerted so that the clinician has the opportunity to perform remedial actions. The clinician may be notified via the computing device with which the clinician is interacting (e.g., the device used to view patient medical images) or by another mechanism. In some embodiments, the clinician is notified during the procedure rather than after, so that the clinician can immediately address any potential or actual issues. After a clinician addresses an issue, the clinician's actual user actions may continue to be logged, obtained, and analyzed in accordance with present embodiments, thereby ensuring that subsequent deviations in the procedure may be detected.

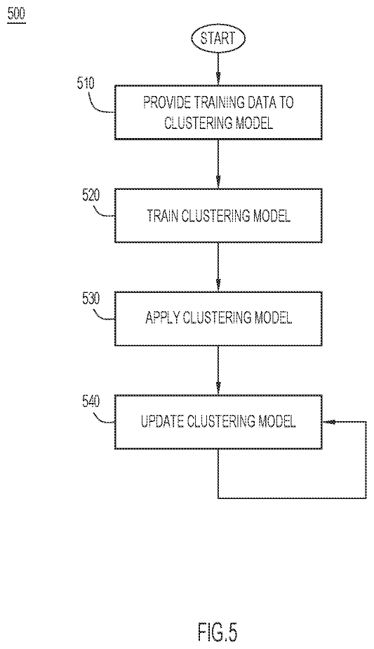

FIG. 5 is a flowchart of a method 500 for training and applying a clustering model according to an embodiment of the present invention.

Training data is provided to a clustering model at operation 510. Training data may include example sets of patient data and corresponding predicted user actions. In various embodiments, the training data includes text data, image data, or both. The data may be labeled with regard to whether the data is screening data, diagnostic data, data specific to an organ, and moreover, the data may be labeled with regard to how the data was obtained (e.g., specific imaging techniques, procedures (e.g., biopsies), and the like). In some embodiments, the training data is unlabeled.

The clustering model is trained at operation 520. The clustering model is trained using the training data until the clustering model is able to identify predicted user actions based on patient data to a satisfactory level of accuracy. In various embodiments, the clustering model may be a k-means clustering algorithm, a density-based spatial clustering of applications with noise (DBSCAN) clustering algorithm, a gaussian mixture model algorithm, a balance iterative reducing and clustering using hierarchies (BIRCH) algorithm, an affinity propagation clustering algorithm, a means-shift clustering algorithm, an ordering points to identify the clustering structure (OPTICS) algorithm, an agglomerative hierarchy clustering algorithm, or another conventional or other clustering algorithm. Training may be completed when the increase in accuracy of the model falls to within a threshold level of improvement from one training cycle to another. The clustering model may include any conventional or other machine learning models (e.g., mathematical/statistical, classifiers, feed-forward, recurrent, convolutional, deep learning, or other neural networks, etc.).

The clustering model is applied at operation 530. The clustering model processes patient data in order to identify predicted user actions. During a procedure, a clinician's actual actions can be compared to the predicted user actions, and outcome data can be obtained during and/or after the procedure. The outcome data may include feedback from the clinician (or other reviewer) indicating whether the predicted user actions were correct or incorrect.

The clustering model is updated at operation 540. The clustering model can be retrained using the outcome data collected from one or more procedures in order to improve the accuracy of the clustering model. Thus, the model may improve in accuracy over time with usage. In some embodiments, the model is updated via unsupervised learning techniques using unlabeled data that includes the actual user actions collected during medical procedures.

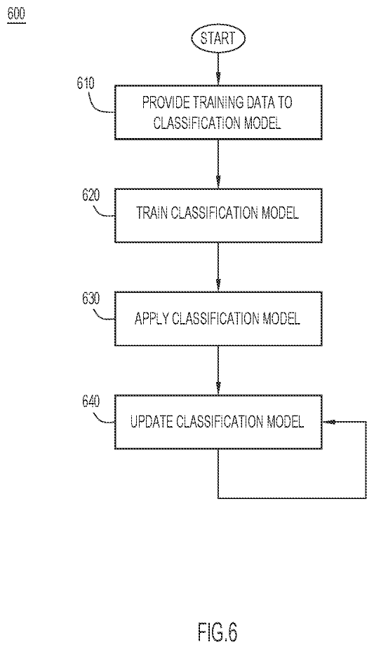

FIG. 6 is a flowchart of a method 600 for training and applying a classification model according to an embodiment of the present invention.

Training data is provided to a classification model at operation 610. Training data may include sets of predicted user actions and actual user actions that are each labeled with respect to whether or not the actual user actions correspond to a deviation in a procedure. In various embodiments, the training data may include text (e.g., commands input by a user), images (e.g., views of images that were displayed to a user) or combinations thereof.

The classification model is trained at operation 620. The model may be trained using the training data until a desired level of accuracy is reached. In various embodiments, the classification model may include a logistic regression model, a decision tree model, a random forest model, a support vector machine, a k-nearest neighbor model, a naïve bayes classifier, or any other conventional or other deep learning model. The classification model may include any conventional or other machine learning models (e.g., mathematical/statistical, classifiers, feed-forward, recurrent, convolutional, deep learning, or other neural networks, etc.).

The classification model is applied at operation 630. The classification model is provided input that includes predicted user actions and actual user actions, and analyzes the data to identify whether a deviation in a procedure has occurred. Outcome data may be collected in the form of feedback from a clinician or other observer indicating whether any detected deviations were correct or incorrect.

The classification model is updated at operation 640. The model can be retrained using the outcome data to improve the accuracy of the model. Thus, future classifications may improve in accuracy as a classification model is employed over time. In some embodiments, the model can be updated via unsupervised learning techniques using data corresponding to actual user actions performed during the procedure.

It will be appreciated that the embodiments described above and illustrated in the drawings represent only a few of the many ways of implementing embodiments for assessing medical procedures for completeness based on machine learning.

The environment of the present invention embodiments may include any number of computer or other processing systems (e.g., any computing device, medical imaging device, computing service, etc.) and databases or other repositories arranged in any desired fashion, where the present invention embodiments may be applied to any desired type of computing environment (e.g., cloud computing, client-server, network computing, mainframe, stand-alone systems, etc.). The computer or other processing systems employed by the present invention embodiments may be implemented by any number of any personal or other type of computer or processing system. These systems may include any types of monitors and input devices (e.g., keyboard, mouse, voice recognition, etc.) to enter and/or view information.

It is to be understood that the software of the present invention embodiments (e.g., patient data module 210, clustering module 220, event collection module 230, classification module 240, etc.) may be implemented in any desired computer language and could be developed by one of ordinary skill in the computer arts based on the functional descriptions contained in the specification and flowcharts illustrated in the drawings. Further, any references herein of software performing various functions generally refer to computer systems or processors performing those functions under software control. The computer systems of the present invention embodiments may alternatively be implemented by any type of hardware and/or other processing circuitry.

The various functions of the computer or other processing systems may be distributed in any manner among any number of software and/or hardware modules or units, processing or computer systems and/or circuitry, where the computer or processing systems may be disposed locally or remotely of each other and communicate via any suitable communications medium (e.g., LAN, WAN, Intranet, Internet, hardwire, modem connection, wireless, etc.). For example, the functions of the present invention embodiments may be distributed in any manner among the various end-user/client, distributed computing, and server systems, and/or any other intermediary processing devices. The software and/or algorithms described above and illustrated in the flowcharts may be modified in any manner that accomplishes the functions described herein. In addition, the functions in the flowcharts or description may be performed in any order that accomplishes a desired operation.

The communication network may be implemented by any number of any type of communications network (e.g., LAN, WAN, Internet, Intranet, VPN, etc.). The computer or other processing systems of the present invention embodiments may include any conventional or other communications devices to communicate over the network via any conventional or other protocols. The computer or other processing systems may utilize any type of connection (e.g., wired, wireless, etc.) for access to the network. Local communication media may be implemented by any suitable communication media (e.g., local area network (LAN), hardwire, wireless link, Intranet, etc.).

The system may employ any number of any conventional or other databases, data stores or storage structures (e.g., files, databases, data structures, data or other repositories, etc.) to store information. The database system may be implemented by any number of any conventional or other databases, data stores or storage structures (e.g., files, databases, data structures, data or other repositories, etc.) to store information. The database system may be included within or coupled to the computing system. The database systems and/or storage structures may be remote from or local to the computer or other processing systems, and may store any desired data.

The present invention embodiments may employ any number of any type of user interface (e.g., Graphical User Interface (GUI), command-line, prompt, etc.) for obtaining or providing information (e.g., resource provider information, requests for resources, job and/or task information, etc.), where the interface may include any information arranged in any fashion. The interface may include any number of any types of input or actuation mechanisms (e.g., buttons, icons, fields, boxes, links, etc.) disposed at any locations to enter/display information and initiate desired actions via any suitable input devices (e.g., mouse, keyboard, etc.). The interface screens may include any suitable actuators (e.g., links, tabs, etc.) to navigate between the screens in any fashion.

Patient data, outcome data, and predicted and actual user action data may include any information arranged in any fashion, and may be configurable based on rules or other criteria to provide desired information to a user (e.g., a clinician).

The present invention embodiments are not limited to the specific tasks or algorithms described above, but may be utilized for assessing procedures for completeness in any computing environments.

The patient data may include any format of storing data, including text-based formats, image-based formats, or other types of media, and may include any data having any relevance to a patient's medical condition. The patient data may be obtained via any techniques, including known or other medical imaging techniques.

The actual user action data may include any format of storing data, including text-based and/or image-based forms, and may be stored in a time-series manner indicating user actions and times at which the user actions are performed, or an order in which the user actions are performed.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises”, “comprising”, “includes”, “including”, “has”, “have”, “having”, “with” and the like, when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method of assessing a medical procedure comprising:
receiving input data corresponding to a patient having a medical procedure;
determining a plurality of predicted user actions to be performed during the medical procedure;
comparing a plurality of actual user actions performed during the medical procedure to the plurality of predicted user actions to identify one or more deviations in the medical procedure; and
alerting a user to the one or more deviations.

2. The method of claim 1, wherein a deep learning clustering model is trained to determine the plurality of predicted user actions based on the input data.

3. The method of claim 2, further comprising:
updating the deep learning clustering model based on one or more from a group of:
user feedback relating to the medical procedure, and user interactions performed during the medical procedure.

4. The method of claim 1, wherein a deep learning classification model is trained to identify the one or more deviations.

5. The method of claim 4, further comprising:
updating the deep learning classification model based on one or more from a group of:
user feedback relating to the medical procedure, and user interactions performed during the medical procedure.

6. The method of claim 1, wherein the plurality of actual user actions are extracted from one or more from a group of: a log generated by a medical device, and image data of the medical procedure.

7. The method of claim 1, wherein the medical procedure is a radiological image analysis procedure.

8. A system for assessing a medical procedure comprising:
one or more memories; and
at least one processor coupled to the one or more memories, wherein the at least one processor is configured to:
receive input data corresponding to a patient having a medical procedure;
determine a plurality of predicted user actions to be performed during the medical procedure;
compare a plurality of actual user actions performed during the medical procedure to the plurality of predicted user actions to identify one or more deviations in the medical procedure; and
alert a user to the one or more deviations.

9. The system of claim 8, wherein a deep learning clustering model is trained to determine the plurality of predicted user actions based on the input data.

10. The system of claim 9, wherein the at least one processor is further configured to:
update the deep learning clustering model based on one or more from a group of: user feedback relating to the medical procedure, and user interactions performed during the medical procedure.

11. The system of claim 8, wherein a deep learning classification model is trained to identify the one or more deviations.

12. The system of claim 11, wherein the at least one processor is further configured to:
updating the deep learning classification model based on one or more from a group of:
user feedback relating to the medical procedure, and user interactions performed during the medical procedure.

13. The system of claim 8, wherein the plurality of actual user actions are extracted from one or more from a group of: a log generated by a medical device, and image data of the medical procedure.

14. The system of claim 8, wherein the medical procedure is a radiological image analysis procedure.

15. A computer program product for performing a workload with dynamic resource adjustment, the computer program product comprising one or more computer readable storage media having program instructions collectively stored on the one or more computer readable storage media, the program instructions executable by at least one processor to cause the at least one processor to:
receive input data corresponding to a patient having a medical procedure;
determine a plurality of predicted user actions to be performed during the medical procedure;
compare a plurality of actual user actions performed during the medical procedure to the plurality of predicted user actions to identify one or more deviations in the medical procedure; and
alert a user to the one or more deviations.

16. The computer program product of claim 15, wherein a deep learning clustering model is trained to determine the plurality of predicted user actions based on the input data.

17. The computer program product of claim 16, wherein the program instructions further cause the at least one processor to:
update the deep learning clustering model based on one or more from a group of: user feedback relating to the medical procedure, and user interactions performed during the medical procedure.

18. The computer program product of claim 15, wherein a deep learning classification model is trained to identify the one or more deviations.

19. The computer program product of claim 18, wherein the program instructions further cause the at least one processor to:
updating the deep learning classification model based on one or more from a group of:
user feedback relating to the medical procedure, and user interactions performed during the medical procedure.

20. The computer program product of claim 15, wherein the plurality of actual user actions are extracted from one or more from a group of: a log generated by a medical device, and image data of the medical procedure.
